(19) United States
(12) Patent Application Publication
(10) Pub. No.: US 2024/0315840 A1
(43) Pub. Date: Sep. 26, 2024

(54) LOW PROFILE BALLOON CATHETER DELIVERY SYSTEM

(71) Applicant: MEDTRONIC VASCULAR, INC., Santa Rosa, CA (US)

(72) Inventors: Marc A. Anderson, Barna (IE); Michael A. Gloss, Minneapolis, MN (US)

(21) Appl. No.: 18/189,296

(22) Filed: Mar. 24, 2023

(51) Int. Cl. A61F 2/24; A61M 25/09

(52) U.S. Cl. CPC A61F 2/2433; A61F 2/2418; A61M 25/09; A61M 2025/09008

(57) ABSTRACT

A method include of delivering a heart valve prosthesis includes loading the heart valve prosthesis in a crimped configuration within a dilator shaft, tracking the dilator shaft within a vasculature of a patient to a desired site with the heart valve prosthesis loaded therewithin, advancing a delivery device through the dilator shaft to the distal end of the dilator shaft, advancing the delivery device through the heart valve prosthesis to load the heart valve prosthesis onto the delivery device, advancing the delivery device with the heart valve prosthesis mounted thereon past the distal end of the dilator shaft to a treatment site within the vasculature of the patient, and deploying the heart valve prosthesis from the delivery device at the treatment site.

### FIELD OF THE INVENTION

The present invention generally relates to endovascular devices and methods, for example, devices for treatment of a diseased artery.

### BACKGROUND

Delivery systems for transcatheter heart valve prostheses may in some cases have a large diameter or crossing profile. Such large delivery systems may result in access site difficulties, tracking/steering difficulty, and/or requiring large diameter access site (e.g., requiring a large diameter femoral vessel) that can limit patient population. These difficulties are particularly apparent near the access site, where the vessels are typically smaller than the vessels as the delivery system nears the heart. Thus, there is a need for a low profile delivery system for percutaneous delivery of transcatheter heart valve prostheses.

### BRIEF SUMMARY OF THE INVENTION

In accordance with a first example hereof, a method of delivering a transcatheter heart valve prosthesis includes loading the transcatheter heart valve prosthesis in a crimped configuration within a dilator shaft of a dilator, tracking the dilator shaft within a vasculature of a patient such that a distal end of the dilator shaft is located at a desired site with the transcatheter heart valve prosthesis loaded therewithin in the crimped configuration, advancing a delivery device through the dilator shaft to the distal end of the dilator shaft, advancing the delivery device through a central lumen of the transcatheter heart valve prosthesis such that the transcatheter heart valve prosthesis is loaded onto the delivery device, advancing the delivery device with the transcatheter heart valve prosthesis mounted thereon past the distal end of the dilator shaft to a treatment site within the vasculature of the patient, and deploying the transcatheter heart valve prosthesis from the delivery device at the treatment site.

In a second example, in the method according to any of the previous or subsequent examples herein, the delivery device is a balloon catheter, and advancing the delivery device through a central lumen of the transcatheter heart valve prosthesis such that the transcatheter heart valve prosthesis is loaded onto the delivery device comprises the transcatheter heart valve prosthesis being loaded onto a balloon of the balloon catheter, and deploying the transcatheter heart valve prosthesis from the delivery device comprises inflating the balloon to radially expand the transcatheter heart valve prosthesis at the treatment site.

In a third example, the method according to any of the previous or subsequent examples herein further comprises after the transcatheter heart valve prosthesis is loaded onto the balloon, partially expanding the balloon such that a first portion of the balloon distal of the transcatheter heart valve prosthesis and a second portion of the balloon proximal of the transcatheter heart valve prosthesis expand to form a dog-bone shaped balloon.

In a fourth example, in the method according to any of the previous or subsequent examples herein, loading the transcatheter heart valve prosthesis in a crimped configuration within the dilator shaft comprises using a dilator insert, the dilator insert having a dilator insert shaft, a distal end of which pushes the transcatheter heart valve prosthesis through the dilator shaft.

In a fifth example, in the method according to any of the previous or subsequent examples herein, the dilator shaft is a peelable dilator shaft, and as the delivery device is advanced through the peelable dilator shaft, an outer shaft of the delivery device splits the peelable dilator shaft proximal of where the transcatheter heart valve prosthesis is located in the peelable dilator shaft.

In a sixth example, in the method according to any of the previous or subsequent examples herein, as the delivery device with the transcatheter heart valve prosthesis mounted thereon is advanced past the distal end of the dilator shaft to the treatment site, the delivery device splits the peelable dilator shaft where the transcatheter heart valve prosthesis is located in the peelable dilator shaft and distal of where the transcatheter heart valve prosthesis is located in the peelable dilator shaft.

In a seventh example, in the method according to any of the previous or subsequent examples herein, the desired site to which the distal end of the dilator shaft is advanced with the transcatheter heart valve prosthesis loaded therein is a descending aorta of the patient.

In an eighth example, in the method according to any of the previous or subsequent examples herein, the treatment site is a native aortic valve of the patient.

In a ninth example, in the method according to any of the previous or subsequent examples herein, loading the transcatheter heart valve prosthesis in the crimped configuration within the dilator shaft comprises loading the transcatheter heart valve prosthesis in the crimped configuration within the distal end of the dilator shaft.

In accordance with a tenth example hereof, a delivery system for delivering a transcatheter heart valve prosthesis includes a dilator including a dilator shaft, a transcatheter heart valve prosthesis loaded into a distal end of the dilator shaft in a crimped configuration, and a balloon catheter including a balloon and an inflation lumen in fluid communication with an interior of the balloon. The distal end of the dilator shaft with the transcatheter heart valve prosthesis disposed therein is configured to be advanced to a desired site within a vasculature of a patient. The balloon catheter is configured to be inserted though the dilator shaft and through a central lumen of the transcatheter heart valve prosthesis to load the transcatheter heart valve prosthesis onto the balloon of the balloon catheter. The balloon catheter with the transcatheter heart valve prosthesis loaded therein is configured to be advanced past the dilator to a desired treatment site within the vasculature of the patient.

In an eleventh example, in the delivery system according to any of the previous or subsequent examples herein, the dilator shaft is a peelable dilator shaft, wherein the peelable dilator shaft and the balloon catheter are configured such that advancing the balloon catheter through the peelable dilator shaft causes the peelable dilator shaft to split.

In a twelfth example, in the delivery system according to any of the previous or subsequent examples herein, the peelable dilator shaft includes a peel line, wherein the balloon catheter is configured to split the peelable dilator shaft along the peel line.

In a thirteenth example, in the delivery system according to any of the previous or subsequent examples herein, the desired site to which the distal end of the dilator shaft is configured to be advanced is a descending aorta of the patient.

In a fourteenth example, in the delivery system according to any of the previous or subsequent examples herein, the treatment site is a native aortic valve of the patient.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A illustrates a dilator of a delivery system according to embodiments hereof.

FIG. 1B illustrates a close up view of a proximal end of the dilator of FIG. 1A.

FIG. 1C illustrates a close up view of a distal end of the dilator of FIG. 1A.

FIG. 2A illustrates a transcatheter heart valve prosthesis loaded into the proximal end of the dilator using a dilator insert according to embodiments hereof.

FIG. 2B illustrates the transcatheter heart valve prosthesis loaded into the distal end of the dilator using the dilator insert of FIG. 2A.

FIG. 3A illustrates a delivery device of the delivery system according to embodiments hereof.

FIG. 3B illustrates a close up view of a distal end of the delivery device of FIG. 3A.

FIG. 4 is a block diagram that illustrates a method of using the delivery system according to embodiments hereof.

FIG. 5A illustrates an exemplary vasculature of a patient according to embodiments hereof.

FIG. 5B illustrates a step in the method of FIG. 4, illustrating the delivery device being inserted into a proximal end of a dilator according to embodiments hereof.

FIG. 5C illustrates a step in the method of FIG. 4, illustrating the delivery device advancing through a peelable dilator shaft of the dilator according to embodiments hereof.

FIG. 5D illustrates a step in the method of FIG. 4, illustrating a balloon of the delivery device partially inflated with the transcatheter heart valve prosthesis loaded on the balloon according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery system. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. It should be understood that use of the term “about” also includes the specifically recited number of value.

The terms “proximal” and “distal” herein when used with respect to a delivery system are used with reference to the clinician using the devices. Therefore, “proximal” and “proximally” mean in the direction toward the clinician, and “distal” and “distally” mean in the direction away from the clinician. The terms “proximal” and “distal” herein when used with respect to a medical device when implanted, such as a heart valve prosthesis, are used with reference to the direction of blood flow. Therefore, “proximal” and “proximally” mean in an upstream direction, and “distal” and “distally” mean in a downstream direction. However, when used with respect to the medical device with the medical device in the delivery system, the terms are used in the same manner as the delivery system.

As used herein, the term “generally” and “substantially” mean approximately. When used to describe angles such as “substantially parallel” or “substantially perpendicular” the term “substantially” means within 10 degrees of the angle. When used to describe shapes such as “substantially” or “generally” cylindrical or “substantially” or “generally” tube-shaped or “generally” or “substantially” conical, the terms mean that the shape would appear cylindrical or tube-shaped or conical to a person of ordinary skill in the art viewing the shape with a naked eye.

The delivery system shown and described herein includes, inter alia, a dilator and a delivery catheter. The delivery system is configured to deliver a transcatheter heart valve prosthesis, for example, an aortic heart valve prosthesis, to a desired target site within a vasculature of a patient, e.g. the aortic valve. The transcatheter heart valve prosthesis can be any transcatheter heart valve prosthesis, and generally includes an annular frame or stent defining a central lumen and a valve component comprising one or more leaflets coupled to an interior surface of the frame. The frame of the heart valve prosthesis may include a plurality of struts that define a plurality of openings or cells. In the embodiments described herein, the delivery catheter is a balloon catheter, and thus the transcatheter heart valve prosthesis is a balloon expandable transcatheter heart valve prosthesis. The method described herein includes tracking the transcatheter heart valve prosthesis through an aorta of a patient. However, this is not meant to be limiting, as the delivery system disclosed can be used to deliver any type of transcatheter heart valve prosthesis through any part of a patient's vasculature. The delivery system and method described herein is generally directed to initially advancing the transcatheter heart valve prosthesis separately from the delivery catheter, and mounting the heart valve prosthesis onto the delivery catheter at a larger vessel in the delivery route, such as the aorta.

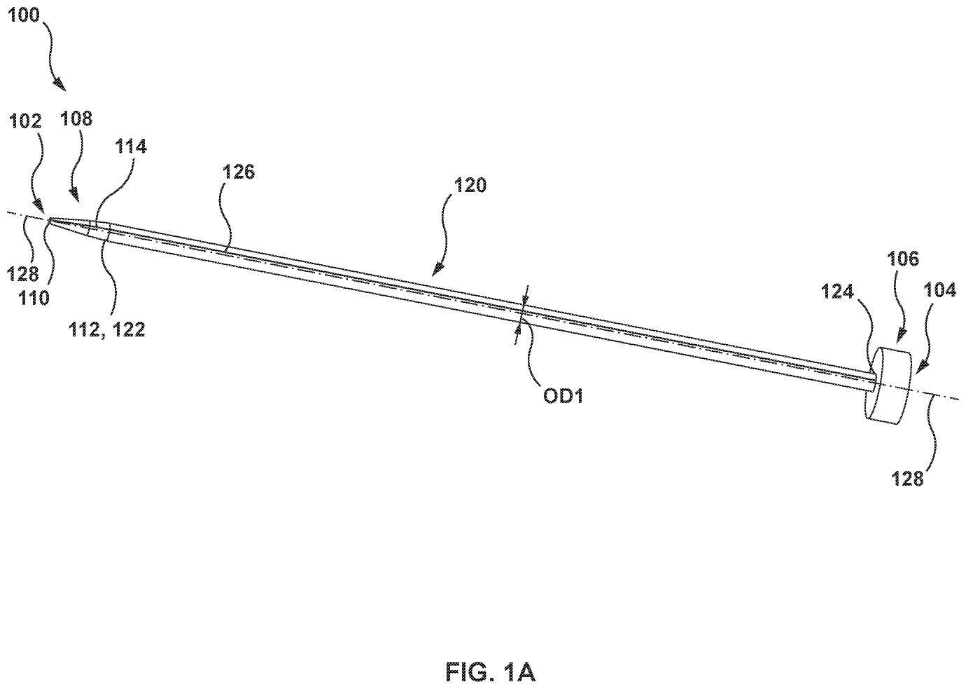

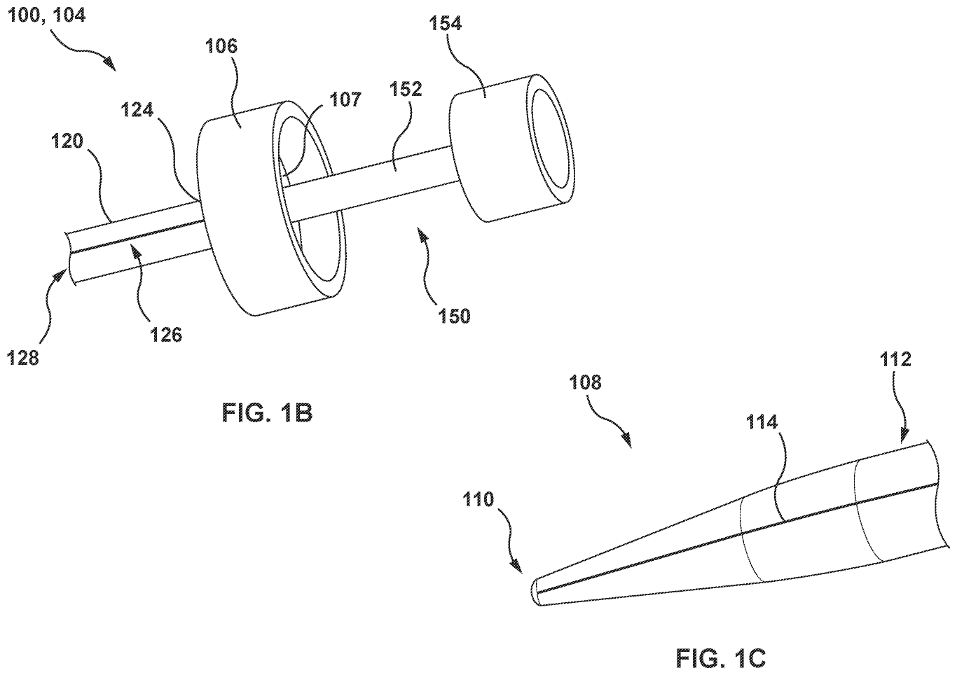

FIG. 1 illustrates a dilator 100 according to embodiments herein. Although referred to as a dilator throughout, the dilator may also be referred to as an introducer. The dilator 100 includes a distal end 102, a proximal end 104, a dilator hub 106, and a peelable dilator shaft 120. The peelable dilator shaft 120 includes a distal end 122 and a proximal end 124. The proximal end 124 of the peelable dilator shaft 120 is coupled to the dilator hub 106 and extends distally therefrom. The distal end 122 of the peelable dilator shaft 120 includes a dilator tip 108. The dilator tip 108 includes a distal end 110, a proximal end 112 and a slot 114. The proximal end 112 of the dilator tip 108 abuts the distal end 122 of the peelable dilator shaft 120, as shown in FIG. 1.

The peelable dilator shaft 120 defines a lumen 128 extending from the proximal end 124 to the distal end 122 of the peelable dilator shaft 120. The peelable dilator shaft 120 further includes a peel line 126 that extends longitudinally from the proximal end 124 to the distal end 122. The peel line 126 may be perforated or comprise less material than the remainder of the peelable dilator shaft 120 such that the peel line is weaker than the remainder of the peelable dilator shaft 120. The peel line 126 is configured to split or peel as a delivery device is advanced through the lumen 128 of the peelable dilator shaft 120, which will be explained in further detail below: The peelable dilator shaft 120 has an outer diameter OD1 of about 5-6 mm. The peelable dilator shaft 120 may have a longitudinal length, from the distal end 122 to the proximal end 124, of about 300-400 mm and may be a polymer or nylon material, such as low density polyethylene (LDPE), high density polyethylene (HDPE), Polyether Block Amide (PEBAX®), or other materials known to those skilled in the art suitable for the purposes described herein, or any combination thereof. The slot 114 of the dilator tip 108 extends from the distal end 110 to the proximal end 112 of the dilator tip 108 and is longitudinally aligned with the peel line 126 of the peelable dilator shaft 120, which is described in further detail below:

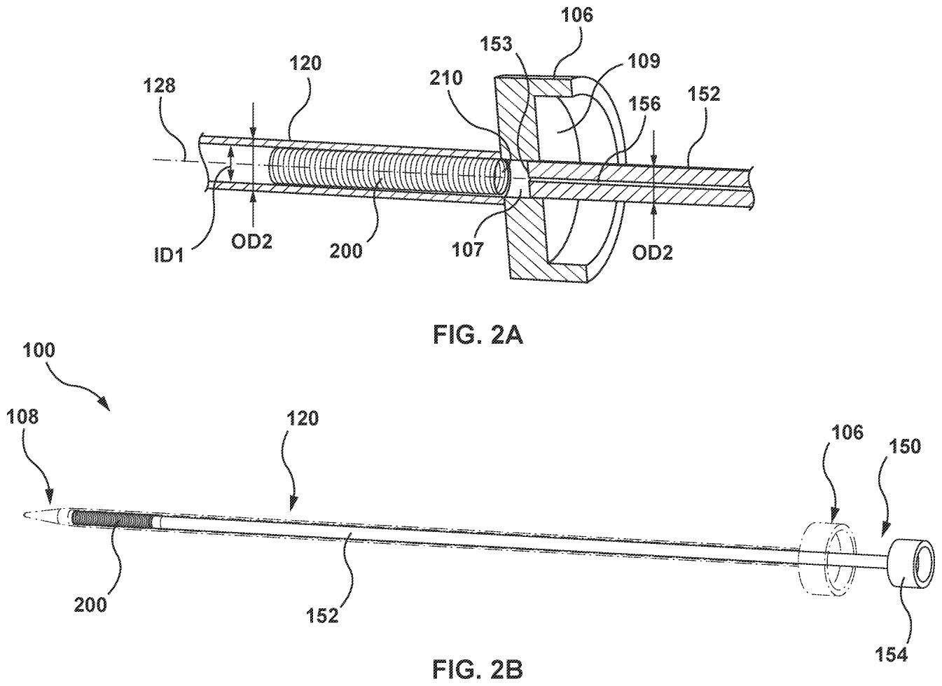

FIG. 1B illustrates a close-up view of the proximal end 104 of the dilator 100. As can be seen, the peelable dilator shaft 120 is coupled to the dilator hub 106 and extends distally therefrom. The peel line 126 of the peelable dilator shaft 120 extends from the proximal end 124 of the peelable dilator shaft 120 to the distal end 122 of the peelable dilator shaft 120 in a substantially straight line. The dilator hub 106 includes a dilator lumen 107 that is in fluid communication with the lumen 128 of the peelable dilator shaft 120, as shown in FIG. 2A. The dilator hub 106 further includes a recessed portion 109 configured to receive a hub or handle of devices inserted through the dilator hub 106. Although not shown, the dilator hub 106 may further include seals and/or valves to prevent leakage of blood out of the peelable dilator shaft 120 through the dilator hub 106. A dilator insert 150 is used to load a transcatheter heart valve prosthesis within the lumen 128 of the peelable dilator shaft 120, which will be described in further detail below: The dilator insert 150 includes a dilator insert shaft 152 and a dilator insert hub 154.

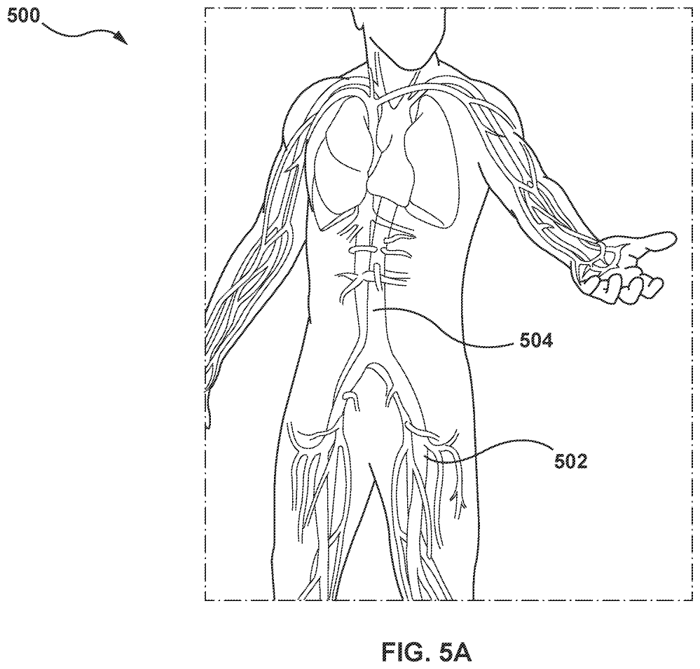

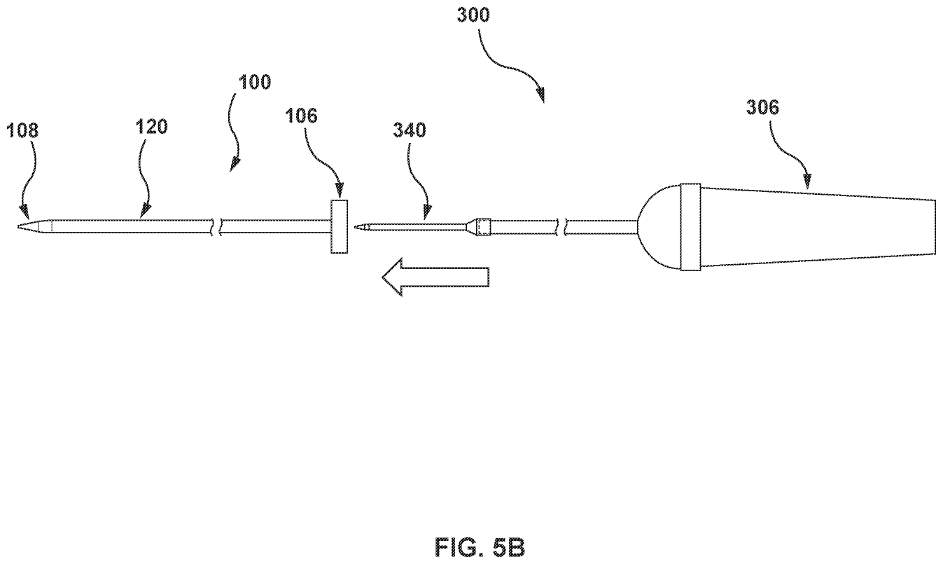

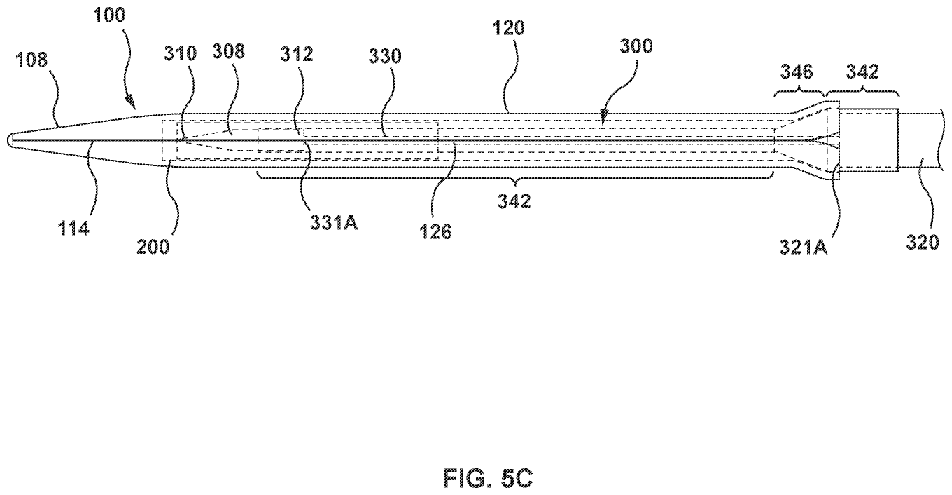

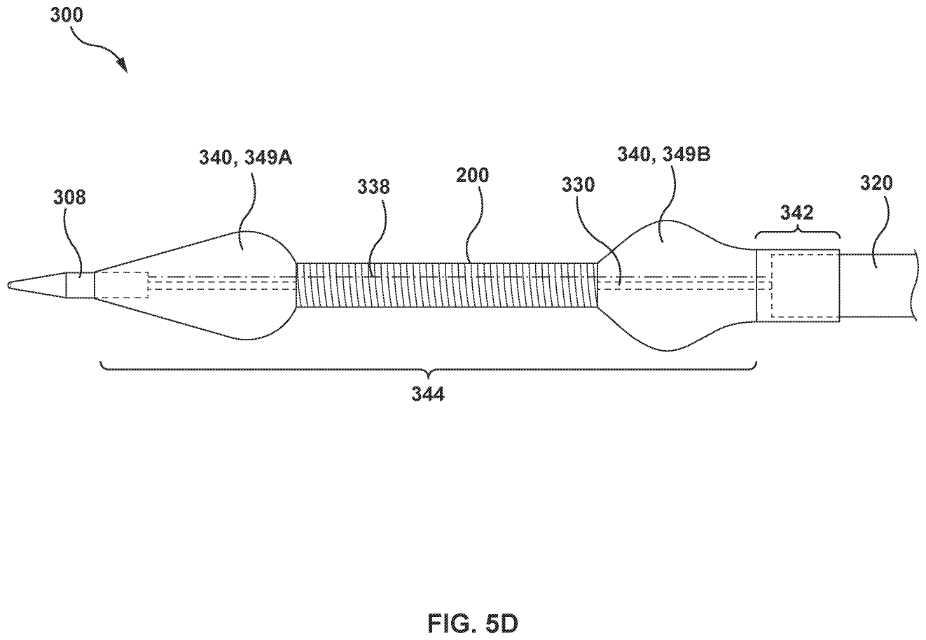

FIG. 1C illustrates a close-up view of the dilator tip 108. The slot 114 of the dilator tip 108 is an opening or gap that extends from the distal end 110 to the proximal end 112 of the dilator tip 108. The slot 114 is longitudinally aligned with the peel line 126 of the peelable dilator shaft 120 of the dilator 100, as best shown in FIG. 5B. The slot 114 is configured to allow the dilator tip 108 to split in a longitudinal direction once a delivery device is advanced through the peelable dilator shaft 120 and has split the entire peel line 126 of the peelable dilator shaft 120, which will be described in further detail below: The dilator tip 108 tapers in a distal direction such that an outer diameter of the proximal end 112 is greater than an outer diameter of the distal end 110.

Prior to delivery within a vasculature of a patient, the transcatheter heart valve prosthesis 200 is loaded within the lumen 128 of the peelable dilator shaft 120 in a crimped configuration. In other words, the transcatheter heart valve prosthesis 200 is compressed and loaded within the peelable dilator shaft 120 of the dilator 100 before the dilator 100 is tracked to a target site within the vasculature of the patient. The dilator insert 150, shown in FIGS. 1B and 2A, is used to load the transcatheter heart valve prosthesis 200 within the lumen 128 of the peelable dilator shaft 120. The dilator insert shaft 152 is coupled to the dilator insert hub 154 and extends distally therefrom. The dilator insert shaft 152 has an outer diameter OD2 that is smaller than the inner diameter ID1 of the peelable dilator shaft 120 such that the dilator insert shaft 152 fits within and may advance through the lumen 128 of the peelable dilator shaft 120 without splitting the peel line 126. To load the transcatheter heart valve prosthesis 200 within the peelable dilator shaft 120 of the dilator 100, the transcatheter heart valve prosthesis 200, in the crimped configuration, is placed at a proximal end of the dilator lumen 107. A distal end 153 of the dilator insert shaft 152 abuts the transcatheter heart valve prosthesis 200 and pushes the transcatheter heart valve prosthesis 200 distally through the dilator hub 106 and through the lumen 128 of the peelable dilator shaft 120 at its proximal end 124, as shown in FIG. 2A. The dilator insert 150 pushes the transcatheter heart valve prosthesis 200 distally within the lumen 128 until the transcatheter heart valve prosthesis 200 reaches the distal end 122 of the peelable dilator shaft 120 and the dilator insert hub 154 abuts the dilator hub 106. The dilator insert shaft 152 may further include a guidewire lumen 156 for a guidewire to pass therethrough, as explained in more detail below. The dilator insert 150 is then proximally withdrawn from the dilator 100 and the transcatheter heart valve prosthesis 200 remains loaded within the distal end 122 of the peelable dilator shaft 120.

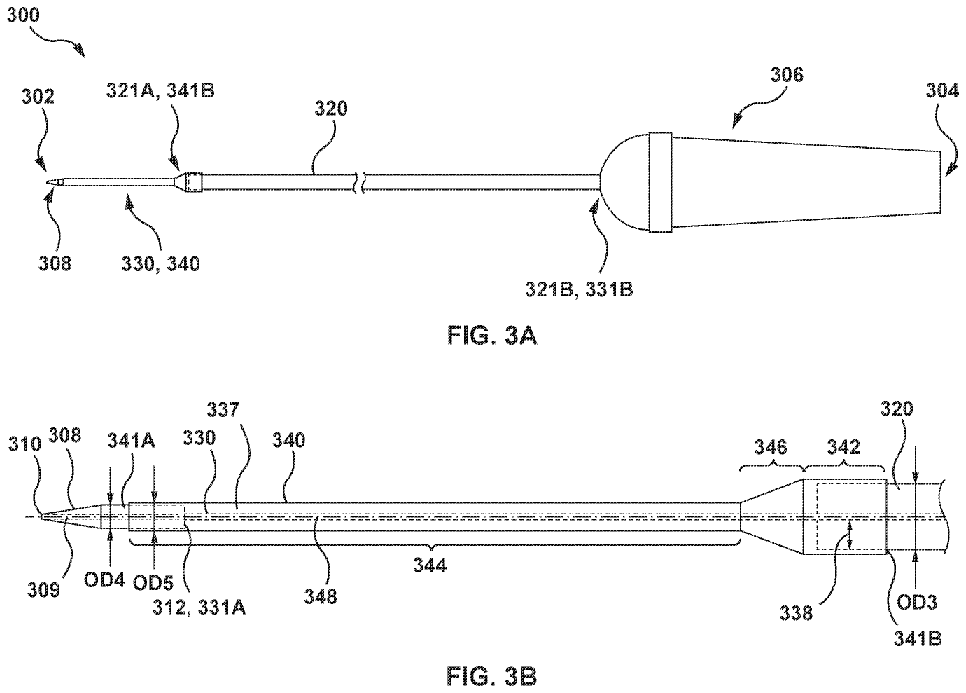

FIGS. 3A-3B illustrate an exemplary delivery device 300 according to embodiments hereof. The delivery device 300 includes a distal end 302, a proximal end 304, a control handle 306, an inner shaft 330, an outer shaft 320 and a tip 308. The outer shaft 320 defines a lumen 328 that extends from a distal end 321A to a proximal end 321B of the outer shaft 320. The inner shaft 330 defines a guidewire lumen 337 that extends from a distal end 331A to a proximal end 331B of the inner shaft. The inner shaft 330 is disposed within the lumen 328 of the outer shaft 320. The proximal ends 321B, 331B of the outer and inner shafts 320, 330 are coupled to the control handle 306 and extend distally therefrom. The outer shaft 320 may have a longitudinal length of about 100 cm-120 cm and the inner shaft 330 may have a longitudinal length of about 110 cm-130 cm such that the distal end 331A of the inner shaft 330 extends distally past the distal end 321A of the outer shaft 320, as shown in FIG. 3A. However, the lengths describe above are not meant to be limiting and are based on femoral access. If other routes are utilized, the lengths may vary. Further, other types of balloon catheters may be utilized. The outer shaft 320 may have an outer diameter OD3 of about 3-5 mm and the inner shaft 330 may have an outer diameter OD4 of about 1-3 mm. The tip 308 of the delivery device 300 includes a distal end 310 and a proximal end 312. The proximal end 312 of the tip 308 is coupled to the distal end 331A of the inner shaft 330 and the tip 308 extends distally therefrom. The tip 308 of the delivery device 300 tapers in a distal direction such that an outer diameter OD4 of the proximal end 312 of the tip 308 is greater than an outer diameter of the distal end 310 of the tip 308. The tip 308 further includes a guidewire lumen 309 extending therethrough and in fluid communication with the guidewire lumen 337 of the inner shaft 330

The delivery device 300 further includes a balloon 340 that is configured to expand the transcatheter heart valve prosthesis 200 upon delivery to the treatment site. The balloon 340 is coupled to a distal portion of the delivery device 300, as shown in FIG. 3A. FIG. 3B illustrates a close-up view of the balloon 340 in FIG. 3A. The balloon 340 includes a distal end 341A, a proximal end 341B, a proximal portion 342, a distal portion 344 and a taper portion 346. In the embodiment shown, the proximal portion 342 of the balloon 340) is substantially cylindrical-shaped and is coupled to an outer surface of the outer shaft 320 of the delivery device 300. The proximal portion 342 of the balloon 340 extends distally about 5-15 mm from the proximal end 341B of the balloon 340 to the taper portion 346 of the balloon 340. The proximal portion 342 of the balloon 340 transitions to the distal portion 344 of the balloon 340 at the taper portion 346. The taper portion 346 is substantially conical-shaped and abuts the proximal portion 342 of the balloon 340 and tapers in a distal direction such that a distal end of the taper portion 346 has an outer diameter that is smaller than an outer diameter of a proximal end of the taper portion 346. The taper portion 346 has a longitudinal length of about 5-15 mm. The distal portion 344 of the balloon 340 abuts the distal end of the taper portion 346 and extends distally about 5-15 mm to the distal end 341A of the balloon 340. The distal portion 344 of the balloon 340 couple to the proximal end 312 of the tip 308 has an outer diameter OD5 of about 2-3 mm. The balloon 340 defines a lumen 348 that extends an entire longitudinal length of the balloon 340, from the distal end 341A to the proximal end 341B. The proximal end 341B of the balloon 340 is coupled to the distal end 321A of the outer shaft 320 of the delivery device 300, as shown in FIG. 3B. The inner shaft 330 extends through the lumen 348 of the balloon 340. The distal end 341A of the balloon 340 is coupled to an outer surface of the tip 308, as shown in FIG. 3B. Thus, the balloon 340 of the delivery device 300 extends from the outer shaft 320 to the tip 308 of the delivery device 300. The inner shaft 330 of the delivery device 300 extends through both the lumen 328 of the outer shaft 320 and through the lumen 348 of the balloon 348 and terminates at the proximal end 312 of the tip 308.

In the embodiment shown, an annular inflation lumen 338 is defined between an outer surface of the inner shaft 330 and an inner surface of the outer shaft 320. The inflation lumen 338 enables an inflation fluid to be delivered to the balloon 340. The inflation fluid travels in the inflation lumen 338 between the outer shaft 320 and the inner shaft 330, exits the inflation lumen 338 through an open distal end of the outer shaft 320, and into the balloon 340 to inflate the balloon 340, as described below. However, this is not meant to be limiting, and other balloon catheter designs with other inflation lumens may be utilized.

In the embodiment shown, the delivery device 300 does not include bumpers. In some balloon catheters in which a transcatheter heart valve prosthesis is loaded onto the balloon of the balloon catheter, bumpers are provided proximal and/or distal of the location of the transcatheter heart valve prosthesis to prevent the transcatheter heart valve prosthesis from moving axially during delivery through the vasculature, i.e., improve valve retention. The bumper(s) may also minimize portions of the frame of the transcatheter heart valve prosthesis, such as crowns thereof, from scraping against the vasculature during delivery through the vasculature. Examples of bumpers can be found, for example, in U.S. Patent Application Publication No. 2022/0054264 A1, which is incorporated by reference herein in its entirety. In embodiments hereof without bumpers, the distal portion of the delivery device 300 may be a smaller profile than if bumpers were included, thereby enabling the delivery system hereof to be used in a larger patient population, as explained below:

The delivery device 300 is configured to be advanced through the dilator 100 when the transcatheter heart valve prosthesis 200 is loaded within the peelable dilator shaft 120 of the dilator 100 and the dilator shaft 120 is positioned within the vasculature of the patient. The outer diameter OD5 of the distal portion 344 of the balloon 340 attached to the tip 308 is sized and shaped to advance through the lumen 128 of the peelable dilator shaft 120 without rupturing the peel line 126 of the peelable dilator shaft 120. Similarly, the outer diameter of the distal portion 344 of the balloon 340 surrounding the inner shaft 330 is sized and shaped to advance through the lumen 128 of the peelable dilator shaft 120 without rupturing the peel line 126 of the peelable dilator shaft 120. In other words, the outer diameter OD4 of the proximal portion 312 of the tip 308 and the outer diameter OD5 of the distal portion 344 of the balloon are smaller than the inner diameter ID1 of the peelable dilator shaft 120 of the dilator 100. Thus, as the tip 308, the inner shaft 330 and the distal portion 344 of the balloon 340 are advanced through the lumen 128 of the peelable dilator shaft 120, the peel line 126 will not split. The outer diameter OD2 of the outer shaft 320 is larger than the outer diameter OD1 of the peelable dilator shaft 120. Thus, as the outer shaft 320 and the proximal portion 342 of the balloon 340 are advanced through the lumen 128 of the peelable dilator shaft 120, the peel line 126 of the peelable dilator shaft 120 will rupture and split, which will be discussed in further detail below.

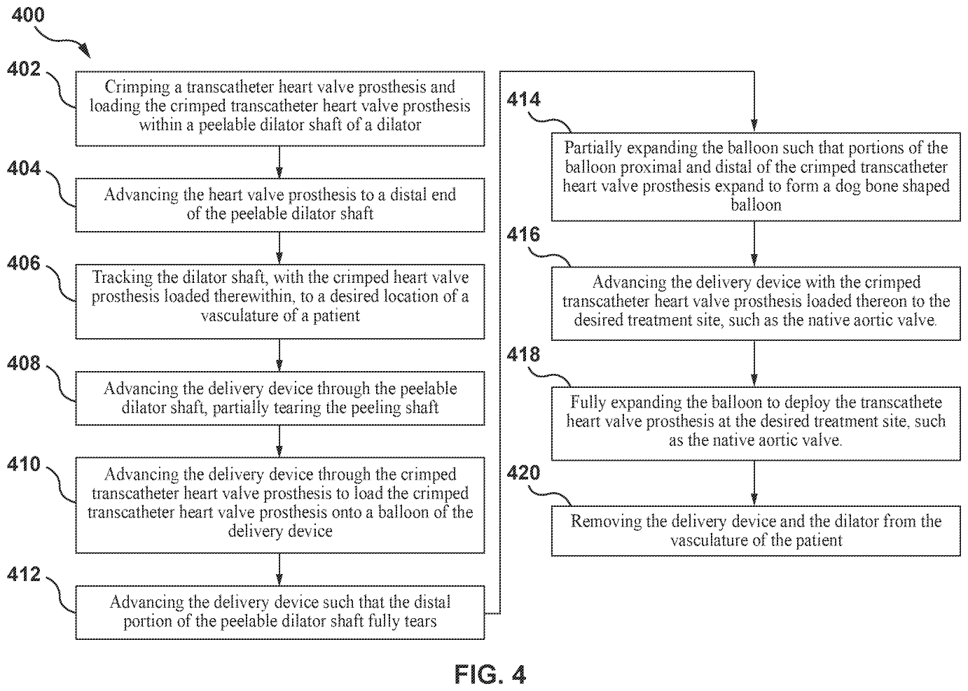

FIG. 4 is a block diagram of a method 400 for delivering a transcatheter heart valve prosthesis 200 within a patient's vasculature 500 using the dilator 100 and the delivery device 300 described above. The transcatheter heart valve prosthesis 200 can be, for example, an aortic heart valve prosthesis. The method described herein will be with respect to delivering an aortic heart valve prosthesis within an aortic valve of a patient, however, this is not meant to be limiting, as the dilator 100 and delivery device 300 described herein can be used to delivery various types of transcatheter prostheses to be deployed in various parts of a patient's vasculature.

In a step 402 of the method 400, the transcatheter heart valve prosthesis 200 is crimped or radially compressed into a crimped configuration. This can be achieved by any method known to those skilled in the art. In embodiments, the transcatheter heart valve prosthesis 200 may be crimped onto a guidewire. In other words, a guidewire is advanced through the central lumen 210 of the transcatheter heart valve prosthesis 200 before the prosthesis 200 is crimped such that when the transcatheter heart valve prosthesis 200 is radially compressed, the guidewire remains disposed within the central lumen 210 of the transcatheter heart valve prosthesis 200. In such an embodiment, the guidewire would also be passed through the guidewire lumen 156 of the dilator insert shaft 152. However, this is not meant to be limiting, as the transcatheter heart valve prosthesis 200 can be crimped without a guidewire disposed within the central lumen 210 of the prosthesis 200. The crimped transcatheter heart valve prosthesis 200 is loaded within lumen 128 of the peelable dilator shaft 120 of the dilator 100, as shown and described with respect to FIGS. 1B, 2A and 2B. The steps of crimping and loading the transcatheter heart valve prosthesis 200 within the lumen 128 of the peelable dilator shaft 120 can be combined such as by using a funnel aligned with the lumen 128 of the peelable dilator shaft 120 such that as the transcatheter heart valve prosthesis 200 is advanced through the funnel, the transcatheter heart valve prosthesis 200 is radially compressed and exits the funnel into central lumen 128 of the peelable dilator shaft 120. However, this is not meant to be limiting, and other methods and devices for crimping and loading the transcatheter heart valve prosthesis 200 into the peelable dilator shaft 120 may be used.

In a step 404 of the method 400, the transcatheter heart valve prosthesis 200 is advanced within the lumen 128 of the peelable dilator shaft 120 to a distal end of the lumen 128, as best shown in FIG. 2B. The transcatheter heart valve prosthesis 200 may be advanced through the lumen 128 using the dilator insert 150, as shown and described above with respect to FIGS. 1B, 2A and 2B, or any other method known to those skilled in the art. When the transcatheter heart valve prosthesis 200 is loaded within the distal end 122 of the peelable dilator shaft 120, the dilator tip 108 is disposed distal of the transcatheter heart valve prosthesis 200. In embodiments, a guidewire is disposed through the lumens 128, 210 of the peelable dilator shaft 120 and transcatheter heart valve prosthesis 200. A proximal end of the guidewire remains outside of the dilator 100 at its proximal end 104. As described above, the peelable dilator shaft 120 has an outer diameter OD1 of about 5-6 mm. Thus, the dilator shaft 120 with the transcatheter heart valve prosthesis 200 loaded therein may be advanced through smaller vessels of the vasculature. This enables a larger number of patient's to be treated with the delivery system disclosed herein. In particular, in some instances, patients with smaller diameter vessels at the entry site for a delivery system may not be able to be treated with traditional delivery systems and methods due to the profile of a delivery device with a transcatheter heart valve prosthesis loaded thereon, such as a balloon catheter with a transcatheter heart valve prosthesis loaded therein.

In a step 406 of the method 400, the dilator shaft 120 with the crimped transcatheter heart valve prosthesis 200 loaded therein is tracked to a desired location within the vasculature 500 of the patient. FIG. 5 shows an exemplary vasculature system 500 of a patient. In an example, the dilator 100 is inserted into the femoral artery 502 and the dilator shaft 120 with the crimped transcatheter heart valve prosthesis 200 loaded therein is tracked to a descending aorta 504 of the patient. In an example, the distal end of a guidewire (not shown) is inserted within the femoral artery 502 of the vasculature 500 and is tracked through the descending aorta 504 to the desired target site, e.g. the aortic valve, while the proximal end of the guidewire remains outside of the vasculature 500. The dilator shaft 120 with the transcatheter heart valve prosthesis 200 disposed therein is advanced over the guidewire and into the vasculature 500 of the patient until the distal end of the dilator shaft 120 reaches the descending aorta 504 within the patient's vasculature 500. The dilator hub 106 and the proximal end of the guidewire remain outside the patient.

In a step 408 of the method 400, the delivery device 300 is advanced through the dilator shaft 120 until the distal end 308 of the delivery device 300 reaches the transcatheter heart valve prosthesis 200 located at the distal end 122 of the dilator shaft 120. In an example, the proximal end of the guidewire (not shown) is inserted into the guidewire lumen 309 of the tip 308 of the delivery device 300, and as the delivery device 300 is advanced, the guidewire passes through the guidewire lumen 337 of the inner shaft 330 and out of the proximal end 304 of the delivery device 300. The distal end 302 of the delivery device 300 is inserted into the lumen 107 of the dilator hub 106 of the dilator 100 and into the lumen 128 of the peelable dilator shaft 120 of the dilator 100, as shown in FIG. 5B.

As the delivery device 300 is advanced through the peelable dilator shaft 120, the outer shaft 320, the tip 308, the distal portion of the inner shaft 330, and the distal portion 344 of the balloon 340 do not rupture the peel line 126 of the peelable dilator shaft 120, as the inner diameter ID1 of the peelable dilator shaft 120 is greater than the outer diameter OD4 of the tip 308 and the outer diameter OD5 of the distal portion 344 of the balloon 340. However, as the outer shaft 320, the distal end 321A of which is proximal to the distal end 331A of the inner shaft 330, the outer shaft 320 of the delivery device 300 will cause the peel line 126 of the peelable dilator shaft 120 to split or rupture, as the outer diameter OD3 of the outer shaft 320 is greater than the outer diameter OD1 of the peelable dilator shaft 120. The peel line 126 of the peelable dilator shaft 120 will split in a substantially straight line along the longitudinal length of the peelable dilator shaft 120 as the outer shaft 320 of the delivery device 300 is advanced through the lumen 128 of the peelable dilator shaft 120, as best shown in FIG. 5C.

In a step 410 of the method 400, the crimped transcatheter heart valve prosthesis 200 is loaded onto the balloon 340 of the delivery device 300 as the delivery device 300 is advanced through the lumen 128 of the peelable dilator shaft 120. The delivery device 300 is advanced distally within the lumen 128 of the peelable dilator shaft 120 until the tip 308 reaches the crimped transcatheter heart valve prosthesis 200. The delivery device 300 continues to be advanced through the lumen 210 of the crimped transcatheter heart valve prosthesis 200 until the tip 308, the distal portion of the inner shaft 330, and the distal portion 342 of the balloon 340 reach a distal side of the crimped transcatheter heart valve prosthesis 200 such that the crimped transcatheter heart valve prosthesis 200 that is disposed on the balloon 340, as shown in FIG. 5C. As the delivery device 300 advances through the central lumen 210 of the transcatheter heart valve prosthesis 200, the outer shaft 320 of the delivery device 300 advances through the lumen 128 of the peelable dilator shaft 120 and splits or peels the peel line 126 starting at the proximal end 124 of the peelable dilator shaft 120. When the peel line 126 splits at the proximal end 124 of the peelable dilator shaft 120, the peelable dilator shaft 120 remains attached to the dilator hub 106 of the dilator 100. As the distal portion 341 of the balloon 340 is advanced through the central lumen 210 of the transcatheter heart valve prosthesis 200, a distal portion of the peelable dilator shaft 120 and the dilator tip 108 remain intact, as the outer shaft 320 of the delivery device 300 has not advanced though the entire peelable dilator shaft 120. The dilator tip 108 provides resistance to hold the transcatheter heart valve prosthesis 200 in place as the delivery device 300 pushes through the central lumen 210 of the transcatheter heart valve prosthesis 200.

In a step 412 of the method 400, the delivery device 300 with the transcatheter heart valve prosthesis 200 centered on the distal portion 342 of the balloon 340 is further advanced through the peelable dilator shaft 120 and the dilator tip 108, thereby splitting the remainder of the peel line 126 of the peelable dilator shaft 120 and the slot 114 of the dilator tip 108. Splitting of the dilator tip 108 along the slot 114 causes the dilator tip 108 to lose its conical shape around the guidewire. Thus, the dilator tip 108 and the peelable dilator shaft 120 detach or fall off the guidewire. The dilator tip 108 and the peelable dilator shaft 120 remain coupled to the dilator hub 106. The dilator tip 108 and the peelable dilator shaft 120 of the dilator 100 remain within the vasculature 500 of the patient since the delivery device 300 is disposed through the dilator hub 106, but the peelable dilator shaft 120 does not obstruct advancement of the delivery device 300.

In a step 412 of the method 400, the balloon 340 of the delivery device 300 is partially expanded with the transcatheter heart valve prosthesis 200 mounted thereon and prior to tracking delivery device 300 to the desired treatment site. To inflate the balloon 340, an inflation fluid is introduced to the delivery device 300 at the proximal end 304. More particularly, the inflation fluid is introduced into the annular inflation lumen 338 defined between the inner shaft 330 and the outer shaft 320 of the delivery device 300, as described above. The fluid is injected within the annular inflation lumen 338 at the proximal end 304 of the delivery device 300 (not shown) and travels distally through the annular inflation lumen 338 to the balloon 340. The inflation fluid exits the inflation lumen 338 at the distal end 321A of the outer shaft 320) and enters into the interior of the balloon 340, thereby inflating the balloon. Because the transcatheter heart valve prosthesis 200 is mounted on the balloon 340, the transcatheter heart valve prosthesis 200 provides greater resistance to expansion of the balloon 340 than portions of the balloon 340 without the transcatheter heart valve prosthesis 200 mounted thereon. Therefore, a first portion 349A of the balloon 340) distal of the transcatheter heart valve prosthesis 200 and a second portion 349B of the balloon 340 to proximal to the transcatheter heart valve prosthesis 200 inflate prior to the central portion of the balloon 340 upon which the transcatheter heart valve prosthesis 200 is mounted, as shown in FIG. 5D. The crimped configuration of the transcatheter heart valve prosthesis 200 prevents the portion of the balloon 340 that it covers from expanding during this partial expansion, but does not prevent fluid from travelling distally to the first portion 349A of the balloon 340. In other words, when the balloon 340 is partially inflated, the first and second portions 349A, 349B of the balloon 340 are expanded, but the transcatheter heart valve prosthesis 200 remains in the crimped configuration on a center of the distal portion 344 of the balloon 340 and does not expand. In other embodiments, other arrangements of inflation shafts/lumens may be provided to assist in delivering the inflation fluid to the first and second portions 349A, 349B of the balloon 340 (particularly the first portion 349A), such as, but not limited to, arrangements described in U.S. Patent Application Publication No. 2020/0383780 A1, which is incorporated by reference herein in its entirety.

As shown in FIG. 5D, the balloon 340 creates a “dog bone” shape with the crimped transcatheter heart valve prosthesis 200 disposed between the inflated first and second portions 349A, 349B of the balloon 340. The inflated first and second portions 349A, 349B of the balloon 340 act as bumpers for valve retention when tracking the delivery device when the balloon 340 is in the partially inflated configuration. The inflated first and second portions 349A, 349B of the balloon 340 also prevent the crowns of the transcatheter heart valve prosthesis 200 from scraping the vasculature 500 of the patient during tracking. Reducing damage to the vasculature can also reduce the occurrence of strokes or other health complications to the patient.

In a step 416 of the method 400, the delivery device 300 with the crimped transcatheter heart valve prosthesis 200 mounted thereon and the first and second portions 349A, 349B of the balloon 340 inflated, is advanced to the desired treatment site, such as the native aortic valve.

In a step 418 of the method 400, with the delivery device 300 at the desired treatment site such that the transcatheter heart valve prosthesis 200 is properly aligned with the native valve, such as the native aortic valve, the balloon 340 is fully inflated to radially expand the transcatheter heart valve prosthesis 200 at the treatment site, e.g., the native aortic valve of the patient. To fully inflate the balloon, additional inflation fluid is introduced to the annular inflation lumen 338 of the delivery device 300. The inflation fluid travels distally to the balloon 340 and expands the balloon 340, including the portion of the balloon 340 that is disposed within the central lumen 210 of the transcatheter heart valve prosthesis 200. The inflation of the balloon 340 causes the transcatheter heart valve prosthesis 200 to radially expand to its expanded configuration.

In a step 420 of the method 400, after the transcatheter heart valve prosthesis 200 has been radially expanded and deployed at the treatment site, the balloon 340 can be deflated by removing the inflation fluid from the balloon 340. The delivery device 300 and the dilator 100 can then be retracted and removed from the patient's vasculature 500.

While the devices and methods have been disclosed herein as used for delivery and deployment of a transcatheter heart valve prosthesis to a native aortic valve, the devices and methods may be used to deliver and deploy transcatheter heart valve prostheses to other native valves, and/or to deliver other devices to other treatment sites. For example, the delivery system may be used in any situation in which it is beneficial to use a low-profile delivery system.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). For example, and not by way of limitations, some steps of the method may be omitted or performed in a different order than presented. In addition, while certain aspects of this disclosure are described as being performed by a single device or component for purposes of clarity, it should be understood that the techniques of this disclosure may be performed by a combination of devices or components.

What is claimed is:

1. A method of delivering a transcatheter heart valve prosthesis, the method comprising:
loading the transcatheter heart valve prosthesis in a crimped configuration within a dilator shaft of a dilator;
tracking the dilator shaft within a vasculature of a patient such that a distal end of the dilator shaft is located at a desired site with the transcatheter heart valve prosthesis loaded therewithin in the crimped configuration;
advancing a delivery device through the dilator shaft to the distal end of the dilator shaft;
advancing the delivery device through a central lumen of the transcatheter heart valve prosthesis such that the transcatheter heart valve prosthesis is loaded onto the delivery device;
advancing the delivery device with the transcatheter heart valve prosthesis mounted thereon past the distal end of the dilator shaft to a treatment site within the vasculature of the patient;
deploying the transcatheter heart valve prosthesis from the delivery device at the treatment site.

2. The method of claim 1, wherein:
the delivery device is a balloon catheter;
advancing the delivery device through a central lumen of the transcatheter heart valve prosthesis such that the transcatheter heart valve prosthesis is loaded onto the delivery device comprises the transcatheter heart valve prosthesis being loaded onto a balloon of the balloon catheter; and
deploying the transcatheter heart valve prosthesis from the delivery device comprises inflating the balloon to radially expand the transcatheter heart valve prosthesis at the treatment site.

3. The method of claim 2, further comprising:
after the transcatheter heart valve prosthesis is loaded onto the balloon, partially expanding the balloon such that a first portion of the balloon distal of the transcatheter heart valve prosthesis and a second portion of the balloon proximal of the transcatheter heart valve prosthesis expand to form a dog-bone shaped balloon.

4. The method of claim 1, wherein loading the transcatheter heart valve prosthesis in a crimped configuration within the dilator shaft comprises using a dilator insert, the dilator insert having a dilator insert shaft, a distal end of which pushes the transcatheter heart valve prosthesis through the dilator shaft.

5. The method of claim 1, wherein the dilator shaft is a peelable dilator shaft, and wherein as the delivery device is advanced through the peelable dilator shaft, an outer shaft of the delivery device splits the peelable dilator shaft proximal of where the transcatheter heart valve prosthesis is located in the peelable dilator shaft.

6. The method of claim 5, wherein as the delivery device with the transcatheter heart valve prosthesis mounted thereon is advanced past the distal end of the dilator shaft to the treatment site, the delivery device splits the peelable dilator shaft where the transcatheter heart valve prosthesis is located in the peelable dilator shaft and distal of where the transcatheter heart valve prosthesis is located in the peelable dilator shaft.

7. The method of claim 1, wherein the desired site to which the distal end of the dilator shaft is advanced with the transcatheter heart valve prosthesis loaded therein is a descending aorta of the patient.

8. The method of claim 7, wherein the treatment site is a native aortic valve of the patient.

9. The method of claim 1, wherein loading the transcatheter heart valve prosthesis in the crimped configuration within the dilator shaft comprises loading the transcatheter heart valve prosthesis in the crimped configuration within the distal end of the dilator shaft.

10. A delivery system for delivering a transcatheter heart valve prosthesis, the delivery system comprising:
a dilator including a dilator shaft;
a transcatheter heart valve prosthesis loaded into a distal end of the dilator shaft in a crimped configuration; and
a balloon catheter including a balloon and an inflation lumen in fluid communication with an interior of the balloon,
wherein the distal end of the dilator shaft with the transcatheter heart valve prosthesis disposed therein is configured to be advanced to a desired site within a vasculature of a patient,
wherein the balloon catheter is configured to be inserted through the dilator shaft and through a central lumen of the transcatheter heart valve prosthesis to load the transcatheter heart valve prosthesis onto the balloon of the balloon catheter, and
wherein the balloon catheter with the transcatheter heart valve prosthesis loaded therein is configured to be advanced past the dilator to a desired treatment site within the vasculature of the patient.

11. The delivery system of claim 10, wherein the dilator shaft is a peelable dilator shaft, wherein the peelable dilator shaft and the balloon catheter are configured such that advancing the balloon catheter through the peelable dilator shaft causes the peelable dilator shaft to split.

12. The delivery system of claim 11, wherein the peelable dilator shaft includes a peel line, wherein the balloon catheter is configured to split the peelable dilator shaft along the peel line.

13. The delivery system of claim 10, wherein the desired site to which the distal end of the dilator shaft is configured to be advanced is a descending aorta of the patient.

14. The delivery system of claim 13, wherein the treatment site is a native aortic valve of the patient.
